(19) United States
(12) Patent Application Publication
(10) Pub. No.: US 2006/0160151 A1
(43) Pub. Date: Jul. 20, 2006

(54) Method of quantitative immunohistochemistry and in situ hybridization

(75) Inventors: D. Craig Allred, Houston, TX (US); Syed Khalid Mohsin, Dublin, OH (US); Carlos Genty, Redondo Beach, CA (US); Sufeng Mao, Houston, TX (US)

Correspondence Address: FULBRIGHT & JAWORSKI, LLP, 1301 MCKINNEY, SUITE 5100, HOUSTON, TX, 77010-3095, US

(73) Assignee: BAYLOR COLLEGE OF MEDICINE, Houston, TX (US)

(21) Appl. No.: 11/329,000

(22) Filed: Jan. 10, 2006

Related U.S. Application Data
(60) Provisional application No. 60/642,606, filed on Jan. 10, 2005.

(51) Int. Cl. G01N 33/53; G01N 33/537; G01N 33/543

(52) U.S. Cl. 435/7.5; 435/7.92

(57) ABSTRACT

The present invention involves a simple and rapid method for quantitative detection of a ligand in a sample using a combination of an ELISA-like assay and immunohistochemical staining.

The present application claims priority to U.S. Provisional Patent Application Ser. No. 60/642,606, filed Jan. 10, 2005, which is incorporated by reference herein in its entirety.

### TECHNICAL FIELD

The present invention relates to methods and compositions for detection and/or identification of a biomolecule in a cell and/or tissue sample. Detection may be accomplished both quantitatively and qualitatively.

### BACKGROUND OF THE INVENTION

Antibodies provide a powerful and versatile means of detecting small amounts of an antigen within a sample and have been used extensively for both research and diagnostic applications. Antibodies can be produced that bind specifically to a desired antigen. A number of schemes have been created for detecting the bound antibody, including the popular enzyme linked immuno-sorbent-assay (ELISA) techniques (Harlow, et al. Antibodies—A Laboratory Manual. Cold Spring Harbor Laboratory (1988)), for example. One method of ELISA involves exposing the antigen-specific antibody to an antigen bound to a substrate. The antibody-antigen complex is exposed to an anti-IgG-Peroxidase complex. A chromogen is added that is oxidized to form a colored product of the peroxidase enzyme, the amount of color being proportional to the amount of peroxidase present that is in turn related to the amount of antigen present. Another ELISA method uses a sandwich assay technique in which a capture enzyme bound to a surface is exposed to an antigen-comprising solution. A second antibody-enzyme complex in which the antibody portion is also directed toward the antigen is added, and this sandwich complex is detected using a chromogen activated by the enzyme.

Most pathological samples are not prepared as frozen tissues but are routinely formalin-fixed and paraffin-embedded (FFPE) to allow for histological analysis and for archival storage. Because paraffin-embedded samples are widely available, rapid and reliable methods are needed for the quantitative detection of protein from such samples. Techniques for the quantification of protein from paraffin-embedded tissues are particularly needed for the study of protein expression in tumor tissues. For example, expression levels of certain receptors or enzymes can indicate the likelihood of success of a particular treatment. Further, rapid techniques for in situ hybridization of intact tissue samples are useful for diagnosis and tracking of disease.

### BRIEF SUMMARY OF THE INVENTION

The present invention generally concerns quantitative immunohistochemistry (IHC) and in situ hybridization and provides novel methods and compositions therefor. In particular, the present invention regards quantitative detection of a binding agent to a ligand. In certain embodiments of the invention, there is quantitative ELISA-like immunohistochemistry on fixed tissue of any kind, including formalin-fixed paraffin-embedded tissue.

The present invention can be practiced with any molecule that can identify a specific ligand (e.g. protein as a non-limiting example) and, in specific embodiments, its covalent/structural form. The present invention may also be performed in multiplex mode such that simultaneous quantitative measurement of the levels of multiple ligands from samples can be performed. The invention thus quantifies the amount of various selected antigens with optionally cellular resolution. The invention may be practiced with a plurality of binding agents, such as about 2-5, about 7, about 10, about 15, about 20, about 25, about 30, about 35, about 40, about 45, about 50, about 55, about 60, about 65, about 70, about 75, about 80, about 85, about 90, or even about 100 (or more) different binding agents (such as antibodies), with each recognizing a specific antigen. A plurality of ligands may thus be simultaneously detected.

The present invention combines routine IHC and ELISA into a single assay with the ability to simultaneously localize and measure multiple proteins (and other molecules, such as RNA and/or DNA, for example) in a highly amplified and quantitative manner (in liquid phase) on histologically intact tissue (e.g. formalin-fixed paraffin-embedded tissue (FFPET) using inexpensive and readily available reagents and supplies. The invention is useful for measuring proteins and other molecules on preserved intact tissues, including but not limited to FFPET. The intact target tissue may be whole histological sections or, alternatively, specific types of cells isolated from histological sections by technologies such as laser capture microdissection, for example. The latter approach provides the added advantages of precisely defining and/or enumerating the cell population being evaluated.

Practice of the present invention with the use of an association of a binding agent to a ligand may be exemplified as follows: contacting a ligand with a component of the binding agent to form a first complex; contacting the first complex with a linker molecule capable of binding both the binding agent and biotin to form a second complex; detecting the complexed binding agent by measurement of an appropriate marker, such as substrate production. As will be appreciated by those skilled in the art, the above acts may be performed in other sequences or combinations, such as, but not limited to, forming a complex of the linker molecule and a biotinylated nucleic acid molecule before contacting the complex with the first complex described above. The skilled artisan would also appreciate that where an antibody is used as the binding agent, one example of a linker molecule would comprise a streptavidin-protein A chimeric (which may also be referred to as a fusion) protein wherein the streptavidin would bind the biotinylated nucleic acid molecule while the protein A would bind the antibody.

It is contemplated that the present invention will be useful for measuring proteins in human, animal, and/or plant tissues using antibodies or antibody-like molecules (through epitope binding) to localize, amplify, and quantify the signal. The proteins contemplated for measurement and quantification in the present invention include prognostic factors in cancer, predictive factors in cancer, proteins involved in general biological pathways, and/or proteins that are structural elements. Additionally, it is contemplated that the present invention is useful for measuring RNA in human, animal, and/or plant tissues using sequence-specific primers and/or probes (through in situ hybridization and/or PCR in situ hybridization, for example) to localize, amplify, and quantify the signal in manner conceptually analogous to that described for proteins. The RNA sequences contemplated for measurement and quantification in the present invention include prognostic factors in cancer, predictive factors in cancer, proteins involved in general biological pathways, and mutated sequences. Additionally, it is contemplated that the present invention is useful for measuring DNA in human, animal, and/or plant tissues using sequence-specific primers and/or probes (through in situ hybridization and/or PCR in situ hybridization) to localize, amplify, and quantify the signal in manner conceptually analogous to that described for proteins. The DNA sequences contemplated for measurement and quantification in the present invention include prognostic factors in cancer, predictive factors in cancer, proteins involved in general biological pathways, SNPs, allelic imbalances, and/or mutated sequences.

For example, a sample may be analyzed according to the method of the present invention. Data may be obtained from microscopic visualization, and the percent positive cells per sample for a ligand may be determined. The percentage of positive cells is correlated with data obtained by quantitative measurement of a ligand, such as optical density, in order to quantitatively detect a ligand in a sample.

In one embodiment, there is a method for quantitatively detecting a ligand in a sample comprising: contacting the sample with a binding agent capable of binding the ligand; contacting a section of the sample with a insoluble indicator, wherein the insoluble indicator allows for microscopic visualization of the binding agent; contacting another section of the sample with a soluble indicator, wherein the soluble indicator allows for quantitative measurement of the binding agent; and correlating the microscopic visualization of the binding agent with the quantitative measurement of the binding agent in order to quantitatively detect the ligand in the sample. In specific embodiments, the sample is a tissue section, a cytospin, or a cell smear. In further specific embodiments, the tissue section is embedded in a solid medium, such as paraffin or plastic. In specific embodiments, the binding agent comprises at least one antibody, such as a primary antibody and/or a secondary antibody, for example. In additional specific embodiments, the secondary antibody is enzyme-linked, such as wherein the enzyme is horseradish peroxidase or alkaline phosphatase. The primary antibody may be biotinylated, in certain aspects of the invention.

In particular embodiments of the invention, the quantitative measurement of the soluble indicator comprises detection of optical density, fluorescence, and/or luminescence. In a specific embodiment, the quantitative measurement of the soluble indicator comprises real-time detection, and in further embodiments, the soluble indicator is a calorimetric, chemifluorescent, and/or chemiluminescent enzyme substrate. Exemplary soluble indicators include a chromogen. Further exemplary soluble indicators include 3-3′,5,5′-tetramethylbenzidine, O-dianisidine, and p-nitrophenyl phosphate, for example. The insoluble indicator may be a chromogen. In specific embodiments, the insoluble indicator comprises 3,3′-diaminobenzidine, 3-amino-9-ethylcarbazole, 4-chloro-1-naphthol, p-phenylenediamine, dihydrochloride/pyrocatechol, naphthol AS-MX phosphate, new fuchsin, AS-BI phosphate, naphthol AS-TR phosphate and 5-bromo-4-chloro-3-indoxyl phosphate (BCIP) Fast Red LB, Fast Garnet GBC, Nitro Blue Tetrazolium (NBT) and/or iodonitrotetrazolium Violet (INT), for example.

In particular aspects of the invention, the binding agent comprises a nucleic acid probe. In other aspects of the invention, the ligand is a polypeptide, a nucleic acid, a lipid, a carbohydrate, or a portion or domain or epitope thereof. In specific embodiments, the ligand is selected from the group consisting of p53, Leu-M1, Mac387, cleaved-caspase 3, mitosin, KP1, topoisomerase II, p27, CD31, cytokeratin, MIBI, Bc12, tubulin, CD3, CD45, and CD20.

In another embodiment of the invention, there is a method of screening a sample, such as a patient sample, for the presence of a tumor-associated marker comprising contacting the sample with a binding agent capable of binding the tumor-associated marker; contacting a section of the sample with a insoluble indicator, wherein the insoluble indicator allows for microscopic visualization of the binding agent; contacting another section of the sample with a soluble indicator, wherein the soluble indicator allows for quantitative measurement of the binding agent; and correlating the microscopic visualization of the binding agent with the quantitative measurement of the binding agent in order to quantitatively detect the tumor-associated marker in the sample.

In a further embodiment of the invention, there is a method for quantitative detection of a ligand in a formalin-fixed paraffin-embedded biological tissue sample comprising: deparaffinizing the sample; heating the sample; detecting the ligand by an enzyme-linked immunoassay in a section of the sample; detecting the ligand by immunohistochemical staining in another section of the sample; and quantitatively detecting the ligand by correlating the results of the immunohistochemical staining with the results of the enzyme-linked immunoassay.

In additional embodiments of the invention, there is one or more kits suitable for performing any method of the invention, such as a kit comprising a soluble indicator, an insoluble indicator, and/or a binding agent, for example.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings.

FIGS. 1A-1C illustrate the general principle of quantitative immunohistochemistry/in situ hybridization (QUELI). FIG. 1A illustrates the procedure where utilizing routine microtomy, serial sections (3-4 μm thick) of a core (2-3 mm in diamter) of formalin-fixed paraffin embedded tissue (FFPET) are placed individually into water-filled wells of a microtiter plate. FIG. 2A shows that the water is evaporated, allowing sections to descend and adhere to the bottom of the wells. The sections are then deparaffinized, followed by heat-induced epitope unmasking (e.g. 110° C. for 30 minutes). Thereafter, the plate is subjected to a series of reagents and washes following a standard ELISA-like procedure. FIG. 1C shows at the primary antibody step of the procedure, a primary antibody is plated into 4 consecutive wells. At the chromogen step of the procedure, the first well for each antibody received an insoluble chromogen (e.g. H₂O₂/DAB). The remaining three wells receive a liquid-phase soluble chromogen (e.g. H₂O₂/TMB) which is allowed to continuously react until a prominent colored signal is generated (e.g. 30 minutes). The continuous generation of colored product greatly amplifies the signal until the reaction is stopped. The wells with DAB receive a hematoxylin counterstain and a liquid coverslip, enabling direct microscopic visualization. The wells with TMB are quantified by measuring optical density (OD) at 450 run wavelength with a standard microplate reader.

FIGS. 2A-2D illustrate the stages of the procedure of quantitative immunohistochemistry/in situ hybridization. FIG. 2A shows a paraffin block that was constructed for analysis. FIG. 2B shows histotechnologist placing serial sections of tissue cute from the core individually into water-filled wells of a microtiter plate. FIG. 2C shows the appearance of the plate just before the chromogen step of the procedure. The wells are clear at this point. FIG. 2D shows the wells after the chromogen step. Each of the three groups contains 8 antibodies arranged in 4 columns and 8 rows. The first column in each group received an insoluble chromogen to enable direct microscopic visualization of the tissue/cell source of the protein recognized by the antibody in the series of 4 wells in a row (see FIG. 3). The next 3 wells in the group received soluble chromogen, resulting in a highly amplified signal which is measured and quantified by reading optical density with a standard microplate reader (see Table 1).

FIG. 3 illustrates 12 exemplary proteins measured in a 96-well microtiter plate using a 2 mm core to FFPET human tonsil, which was chosen because it comprises a variety of cell types and antigens. The results in this panel are photomicrographs taken of intact tissue cores in wells receiving insoluble chromogen, enabling identification of the tissue and/or cells expressing the protein. The positive cells can be counted or otherwise quantified (e.g. by computer-aided image analysis), providing a reference to attribute the signal measured in a more highly quantitative linear manner in the adjacent wells with soluble chromogen.

### DETAILED DESCRIPTION OF THE INVENTION

It is readily apparent to one skilled in the art that various embodiments and modifications can be made to the invention disclosed in this Application without departing from the scope and spirit of the invention.

I. Definitions

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having”, “including”, “containing” and “comprising” are interchangeable and one of skill in the art is cognizant that these terms are open ended terms. Some embodiments of the invention may consist of or consist essentially of one or more elements, method steps, and/or methods of the invention. It is contemplated that any method or composition described herein can be implemented with respect to any other method or composition described herein.

The term “binding agent” refers to any molecule or complex of molecules capable of binding a ligand of the invention under suitable conditions. Preferably, the binding occurs with sufficient specificity to exclude detectable binding to more than one other ligand. Even more preferred are “binding agents” that bind with specific specificity to one ligand such that no detectable binding is observed for other ligands. In preferred embodiments of the invention, the “binding agent” is an antibody or ligand binding fragment or analog thereof. The “binding agent” may also be other proteins or nucleic acids, or portions or analogs thereof, that bind a ligand in the practice of the present invention. A “binding agent” and its cognate ligand may be considered binding pairs, of which non-limiting examples include receptors and their ligands; enzymes and substrates or substrate analogs or pseudo-substrates (substrate analogs that cannot be catalyzed by the enzymatic activity); enzymes and their cofactors or inhibitors or modulators; and nucleic acid and nucleic acid-binding proteins. Methods for the generation of antibodies as binding agents are known in the art. In the practice of the invention, binding agent that remains unbound to ligand of a sample after contact therewith may of course be washed away prior to further treatment(s) of the sample. In certain embodiments of the invention, the binding agent is a primary antibody and an enzyme-linked secondary antibody. In other embodiments of the invention, the binding agent is a nucleic acid molecule, or probe, that is complementary to a ligand that is a nucleic acid molecule. The probe in such embodiments may be detectable, such as comprising a label, for example biotin. In embodiments of the invention where the “binding agent” is an antibody, the ligand may be referred to as an “antigen” that is recognized and bound by the antibody under suitable conditions.

The terms “cell” and/or “tissue sample” include, but are not limited to, a tissue section, specific types of cell isolated from tissue sections (e.g. by laser capture microdissection), a cytospin, a cell smear, or a mixture thereof. The terms encompass samples regardless of their physical condition; stated differently, the terms do not exclude material by virtue of the physical state (such as, but not limited to, being frozen or stained or otherwise treated).

As used herein, “conjugated” or “attached” or “linked” refers to the covalent or non-covalent, as well as direct or indirect, association of a binding agent and a nucleic acid molecule. In preferred embodiments, the nucleic acid molecule is connected to the binding agent covalently, but it may also be attached non-covalently, via, for example, protein-protein interactions (such as, but not limited to, biotin/streptavidin or protein A/antibody interactions) or complexation.

By “correlate” or “correlating” is meant comparing, in any way, the performance and/or results of a first analysis with the performance and/or results of a second analysis. For example, one may use the results of a first analysis in carrying out the second analysis and/or one may use the results of a first analysis to determine whether a second analysis should be performed and/or one may compare the results of a first analysis with the results of a second analysis. In certain embodiments of the invention, data obtained from microscopic visualization, such as percent positive cells, are correlated with data obtained by quantitative measurement of a ligand, such as optical density, in order to quantitatively detect a ligand in a sample.

As used herein, “detect” or “detecting” a ligand refers to finding or discovering the presence or existence of said ligand. Use of “identify” or “identifying” with respect to a ligand refers to determining the identity of said ligand by use of a binding agent that recognizes and binds to the ligand. Detecting a ligand may be qualitative, quantitative, or both. Quantitative measurements contemplated by the present invention include counting cells positive for the ligand and/or measuring concentration of the ligand in a sample, for example.

As used herein, an indicator may be “soluble” or “insoluble.” The solublity of the indicator is dependent on the solvent used in the assay. In preferred embodiments of the invention, the solvent is water. The indicator may be a chromogen, an enzyme substrate, a radioactive isotope, a fluorophore, or a combination thereof.

The term “ligand” refers to a component of a cell or tissue sample, including, but not limited to molecules found in or on, produced by, and/or secreted by a cell. The term encompasses proteins (polypeptides and/or peptides) found in, on or outside a cell as well as a biomolecule such as nucleic acids, lipids, carbohydrates, metabolites, and combinations thereof. In the case of proteins, the term includes proteins of any size and conformation, including multimeric proteins, that may be detected by a binding agent. The term also encompasses a portion or domain or epitope of any protein or other biomolecule that may be detected by a binding agent.

As used herein, “microdissection” or any variation of the term broadly refers to mean separation technique by which portions of a cell containing sample can be separated from other portions of the sample. Non-limiting examples of an appropriate means to isolate some cells from a cell-comprising sample are laser-capture microdissection (LCM), laser microdissection (LMD), laser cutting, and/or manual microdissection. LCM is reviewed, for example, by Emmert-Buck et al. (Science, 1996, 274:998-1001). A method which permits the preparation of one or more than one cell of interest from a cell containing sample may be used in combination of the invention.

“Nucleic acid molecule” refers to a polynucleotide or other polymeric form of nucleotides (ribonucleotides and deoxyribonucleotides) of any length. The term includes double- and single-stranded DNA and RNA as well as DNA/RNA hybrids. It also includes known types of modifications including labels known in the art, methylation, “caps”, substitution of one or more of the naturally occurring nucleotides with an analog, and internucleotide modifications, such as uncharged linkages (e.g., phosphorothioates, phosphorodithioates, etc.), appendant moieties (including proteins such as nucleases, toxins, antibodies, signal peptides, poly-L-lysine, etc.), intercalators (e.g., acridine, psoralen, etc.), chelators (e.g., metals, radioactive metals, etc.), alkylators, modified linkages (e.g., alpha anomeric nucleic acids, etc.), as well as unmodified forms of the polynucleotide.

The term “primary antibody” herein refers to an antibody that binds specifically to the target protein antigen in a tissue sample. A primary antibody is generally the first antibody used in an immunohistochemical (IHC) procedure. In one embodiment, the primary antibody is the only antibody used in an IHC procedure. The term “secondary antibody” herein refers to an antibody that binds specifically to a primary antibody, thereby forming a bridge between the primary antibody and a subsequent reagent, if any. The secondary antibody is generally the second antibody used in an immunohistochemical procedure.

A “sample” of the present invention is of biological origin, in specific embodiments, such as from eukaryotic organisms, but in alternative embodiments could be that of a prokaryotic organism. In preferred embodiments, the sample is a human sample, but animal or plant samples may also be used in the practice of the invention. Non-limiting sources of a sample for use in the present invention include solid tissue, fluidic extracts, blood, plasma, serum, spinal fluid, lymph fluid, the external sections of the skin, respiratory, intestinal, and genitourinary tracts, tears, saliva, milk, tumors, organs, cell cultures and/or cell culture constituents, for example. The present invention is particularly useful for solid tissue samples where the amount of available material is small. The method can be used to examine an aspect of expression of a ligand or a state of a sample, including, but not limited to, comparing different types of cells or tissues, comparing different developmental stages, and detecting or determining the presence and/or type of disease or abnormality.

For the purposes herein, a “section” of a tissue sample regards a single part or piece of a tissue sample, e.g. a thin slice of tissue or cells cut from a tissue sample. It is understood that multiple sections of tissue samples may be taken and subjected to analysis according to the present invention. In some cases, the selected portion or section of tissue comprises a homogeneous population of cells. In other cases, the selected portion comprises a region of tissue, e.g. the lumen as a non-limiting example. The selected portion can be as small as one cell or two cells, or could represent many thousands of cells, for example. In most cases, the collection of cells is important, and while the invention has been described for use in the detection of cellular components, the method may also be used for detecting non-cellular components of an organism (e.g. soluble components in the blood as a non-limiting example).

Without limiting the invention, sample “staining” or “microscopic visualization” may be performed by any means, including, but not limited to, histochemical staining (e.g. hematoxylin and eosin), immunostaining (detection with an antibody followed by an enzymatic reaction yielding a color reaction product), and/or computerized image analysis.

II. The Present Invention

The present invention may be used to detect one or more of a large variety of ligands found in samples comprising one or more cells. In preferred embodiments of the invention, quantification (optionally simultaneous quantification) of biomarkers, such as of the different biomarkers currently used in cancer and other diseases is performed, such as for the purpose of diagnosis, prognosis and/or predictive medicine. The present invention may be used to generate one or more tests that quantify a relative signature of one or more biomarkers. This signature could then be compared to one or more standards, such as in a database in which different signatures of these biomarkers are already correlated with diagnosis, prognosis and predictive medicine for cancer, for example. The standard may be available on the World Wide Web or provided to a health care provider, such as in instructions in a kit, for example.

The methods of the invention may be applied to any type of tissue, including cancer tissue, for example. While frozen tumor tissue is not widely available, paraffin blocks are routinely prepared from every type of tumor after surgery, allowing large-scale retrospective investigations of TS expression and chemotherapy response to be performed. Moreover, the technique can be applied to any of a wide range of tumor types and to an unlimited range of target genes. This has implications for the future preparation of individual tumor “gene expression profiles” whereby expression levels could be determined in individual patient samples for one or more genes, such as for a range of genes, that are known to influence clinical outcome and response to various chemotherapeutic agents.

III. Tissue Samples

Biological samples are often fixed with a fixative. Aldehyde fixatives such as formalin (formaldehyde) and glutaraldehyde are typically used. Tissue samples fixed using other fixation techniques such as alcohol immersion (Battifora and Kopinski, J. Histochem. Cytochem. (1986) 34:1095) are also suitable. The samples used may also be embedded in paraffin. In one embodiment, the samples are both formalin-fixed and paraffin-embedded. In another embodiment, the FFPET block is hematoxylin and eosin stained prior to selecting one or more portions for analysis in order to select specific area(s) for the FFPET core sample.

A. Preparation of Tissue Samples

The method of preparing tissue blocks from these particulate specimens has been successfully used in previous IHC studies of various prognostic factors, and/or is well known to those of skill in the art (see, for example, Brown et al., 1990; Abbondanzo et al., 1990; Allred et al., 1990).

Briefly, any intact organ or tissue may be cut into fairly small pieces and incubated in various fixatives (e.g. formalin, alcohol, etc.) for varying periods of time until the tissue is “fixed”. The samples may be virtually any intact tissue surgically removed from the body. The samples may be cut into reasonably small piece(s) that fit on the equipment routinely used in histopathology laboratories. The size of the cut pieces typically ranges from a few millimeters to a few centimeters.

In one specific embodiment, frozen-sections may be prepared by rehydrating 50 ng of frozen “pulverized” tissue at room temperature in phosphate-buffered saline (PBS) in small plastic capsules; pelleting the particles by centrifugation; resuspending them in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections.

Permanent sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections.

In other embodiments, the present invention may utilize standard frozen samples, such as those that are embedded in OCT and that are not pulverized, for example, including those used in standard Frozen Section hospital labs.

B. Deparaffinization of Samples

Deparaffinization removes the bulk of paraffin from the paraffin-embedded sample. A number of techniques for deparaffinization are known, and any suitable technique can be used with the present invention. The preferred method of the invention utilizes washing with an organic solvent to dissolve the paraffin. Such solvents are able to remove paraffin effectively from the tissue sample without adversely affecting the ligands in the tissue. Suitable solvents can be chosen from exemplary solvents such as benzene, toluene, ethylbenzene, xylenes, and mixtures thereof. A xylene is the preferred solvent for use in the methods of the invention. Solvents alone or in combination in the methods of the invention are preferably of high purity, usually greater than about 99%.

Paraffin is typically removed by washing with an organic solvent, with vigorous mixing followed by centrifugation. Samples are centrifuged at a speed sufficient to cause the tissue to pellet in the tube, usually at about 10,000 to about 20,000×g. After centrifugation, the organic solvent supernatant is discarded. One of skill in the art of histology will recognize that the volume of organic solvent used and the number of washes necessary will depend on the size of the sample and the amount of paraffin to be removed. The greater the amount of paraffin to be removed, the more washes that will be necessary. Typically, a sample will be washed between 1 and about 10 times, and preferably, between about two and about four times. A typical volume of organic solvent is about 500 μL for a 10 μm tissue specimen.

Other methods for deparaffinization known to one of skill in the art may also be used in the method of the invention, including direct melting (Banerjee et al., 1995), for example.

In additional embodiments, citrus-based aliphatic hydrocarbons (D-Limolene based, for example) may be employed, including other exemplary proprietary formulations used for deparaffinization (e.g. Hemo-De® (PMP Medical Industries, Inc., Irving, Tex.); Clear-Rite® (Microm International; Walldorf, Germany); EZ-DEWAX™ (BioGenex, San Ramon, Calif.)), for example. EZ-DEWAX™ is known to be a de-waxing and rehydration agent.

C. Rehydration

Samples may be rehydrated after deparaffinization. The preferred method for rehydration is step-wise washing with aqueous lower alcoholic solutions of decreasing concentration. Ethanol is a preferred lower alcohol for rehydration. Other alcohols may also be suitable for use with the invention including methanol, isopropanol and other similar alcohols in the C1-C5 range. The sample is alternatively vigorously mixed with alcoholic solutions and centrifuged. In a preferred embodiment, the concentration range of alcohol is decreased stepwise from about 100% to about 70% in water over about three to five incremental steps, where the change in solution concentration at each step is usually less than about 10% (i.e., the exemplary sequence: 100%, 95%, 90%, 80%, 70%). In another embodiment, deparaffinization and rehydration are carried out simultaneously using a reagent such as EZ-DEWAX™ (BioGenex, San Ramon, Calif.), for example.

D. Pretreatments

In certain embodiments of the invention, samples may be pretreated, such as to facilitate directly or indirectly the methods of the invention. Pretreatments for making targets available (Heat Induced Epitope Retrieval or Proteolytic Enzyme mediated) may be employed. Citrate Buffers, Tris, and EDTA base may be employed as exemplary heat-induced reagents. Pepsin, Proteinase K, Trypsin, Protease and all of the subtypes may also be employed, in certain aspects of the invention, such as by utilizing the many proprietary formulations available.

IV. Antibody Conjugates

The present invention further provides antibodies against ligand proteins, polypeptides and peptides, generally of the monoclonal type, that are linked to at least one agent to form an antibody conjugate. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules that have been attached to antibodies include toxins, anti-tumor agents, therapeutic enzymes, radio-labeled nucleotides, antiviral agents, chelating agents, cytokines, growth factors, and/or oligo- or poly-nucleotides. By contrast, a reporter molecule is defined as any moiety that may be detected using an assay. Non-limiting examples of reporter molecules that have been conjugated to antibodies include enzymes, radiolabels, haptens, fluorescent labels, phosphorescent molecules, chemiluminescent molecules, chromophores, luminescent molecules, photoaffinity molecules, colored particles and/or ligands, such as biotin.

Any antibody of sufficient selectivity, specificity or affinity may be employed as the basis for an antibody conjugate. Such properties may be evaluated using conventional immunological screening methodology known to those of skill in the art. Sites for binding to biological active molecules in the antibody molecule, in addition to the canonical antigen binding sites, include sites that reside in the variable domain that can bind pathogens, B-cell superantigens, the T cell co-receptor CD4 and the HIV-1 envelope (Sasso et al., 1989; Shorki et al., 1991; Silvermann et al., 1995; Cleary et al., 1994; Lenert et al., 1990; Berberian et al., 1993; Kreier et al., 1991). In addition, the variable domain is involved in antibody self-binding (Kang et al., 1988) and contains epitopes (idiotopes) recognized by anti-antibodies (Kohler et al., 1989).

Certain examples of antibody conjugates are those conjugates in which the antibody is linked to a detectable label. “Detectable labels” are compounds and/or elements that can be detected due to their specific functional properties, and/or chemical characteristics, the use of which allows the antibody to which they are attached to be detected, and/or further quantified if desired.

Many appropriate imaging agents are known in the art, as are methods for their attachment to antibodies (see, for e.g., U.S. Pat. Nos. 5,021,236; 4,938,948; and 4,472,509, each incorporated herein by reference). The imaging moieties used can be paramagnetic ions; radioactive isotopes; fluorochromes; NMR-detectable substances; and/or X-ray imaging, for example.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), and/or gadolinium. Ions useful in other contexts, such as X-ray imaging, include but are not limited to lanthanum (III), gold (III), lead (II), and bismuth (III), for example.

In the case of radioactive isotopes for therapeutic and/or diagnostic application, one might employ astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and/or yttrium⁹⁰. ¹²⁵I is often being preferred for use in certain embodiments, and technicium^{99m} and/or indium¹¹¹ are also often preferred due to their low energy and suitability for long range detection. Radioactively labeled monoclonal antibodies of the present invention may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the invention may be labeled with technetium^{99m} by ligand exchange process, for example, by reducing pertechnate with stannous solution, chelating the reduced technetium onto a Sephadex column, and applying the antibody to this column. Alternatively, direct labeling techniques may be used, e.g., by incubating pertechnate, a reducing agent such as SNCl₂, a buffer solution such as sodium-potassium phthalate solution, and the antibody. Intermediary functional groups that are often used to bind radioisotopes that exist as metallic ions to antibody are diethylenetriaminepentaacetic acid (DTPA) or ethylene diaminetetracetic acid (EDTA).

Exemplary fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Antibody conjugates contemplated in the present invention include those for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase and/or glucose oxidase. Preferred secondary binding ligands are biotin and/or avidin and streptavidin compounds. The use of such labels is well known to those of skill in the art and are described, for example, in U.S. Pat. Nos. 3,817,837; 3,850,752; 3,939,350; 3,996,345; 4,277,437; 4,275,149 and 4,366,241; each incorporated herein by reference.

Molecules containing azido groups may also be used to form covalent bonds to proteins through reactive nitrene intermediates that are generated by low intensity ultraviolet light (Potter & Haley, 1983). In particular, 2- and 8-azido analogues of purine nucleotides have been used as site-directed photoprobes to identify nucleotide binding proteins in crude cell extracts (Owens & Haley, 1987; Atherton et al., 1985). The 2- and 8-azido nucleotides have also been used to map nucleotide binding domains of purified proteins (Khatoon et al., 1989; King et al., 1989; and Dholakia et al., 1989) and may be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. Nos. 4,472,509 and 4,938,948, each incorporated herein by reference). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors, for example, is achieved using monoclonal antibodies, and the detectable imaging moieties are bound to the antibody using linkers such as methyl-p-hydroxybenzimidate or N-succinimidyl-3-(4-hydroxyphenyl)propionate.

In other embodiments, derivatization of immunoglobulins by selectively introducing sulfhydryl groups in the Fc region of an immunoglobulin using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region, have also been disclosed in the literature (O'Shannessy et al., 1987).

In additional embodiments of the invention, nanogold particles (such as sizes from about 0.5 nm-40 nm) and/or Quantum Dots (Hayward, Calif.) are employed.

V. Substrates and Indicators

In certain embodiments of the invention, the use of substrates and indicators is contemplated, such as the exemplary embodiments provided below, for example. In other specific embodiments, beta-galactosidase and/or glucose oxidase detection systems are employed.

A. Horseradish Peroxidase

Horseradish peroxidase (HRP)(molecular weight 40 kD) is isolated from the root of the horseradish plant. HRP has an iron-containing heme group (hematin) as its active site and in solution is colored brown. The hematin of HRP first forms a complex with hydrogen peroxide and then causes it to decompose, resulting in water and atomic oxygen. HRP oxidizes several substances, two of which are polyphenols and nitrates. Like many other enzymes, HRP and some HRP-like activities can be inhibited by excess substrate. The complex formed between HRP and excess hydrogen peroxide is catalytically inactive and in the absence of an electron donor (e.g. chromogenic substance) is reversibly inhibited. It is the excess hydrogen peroxide and the absence of an electron donor that brings about quenching of endogenous HRP activities. Cyanide and azide are two other strong (reversible) inhibitors of HRP. HRP can be attached to other proteins either covalently or noncovalently. The covalent binding of HRP to other proteins can be performed using either one-step or two-step procedures involving glutaraldehyde. The chemical 4,4′-difluoro-3,3′-dinitrophenyl sulfone (FNPS) is less commonly used for this purpose. In all cases, the epsilonamino groups of lysine and N-terminal amino groups of both proteins are involved in this reaction. The two-step conjugation procedure is preferred because, relative to the antibody molecule, the HRP molecule has few reactive groups. As a consequence, adding glutaraldehyde to a solution containing an admixture of HRP and antibody will result in more antibody molecules being conjugated to each other than to the enzyme. In the two-step procedure, HRP reacts with the bifunctional reagents first. In the second stage, only activated HRP is admixed with the antibody, resulting in much more efficient labelling and no polymerization. HRP is also conjugated to (strept)avidin using the two-step glutaraldehyde procedure. This form is used in the LAB and LSAB procedures, for example. Conjugation with biotin also involves two steps, as biotin must first be derivatized to the biotinyl-N-hydroxysuccinimide ester or to biotin hydrazide before it can be reacted with the epsilonamino groups of the enzyme.

Noncovalent binding of HRP to antibody (also known as unlabelled antibody binding) is described in great detail by Sternberger. Instead of the use of bifunctional reagents, IgG-class antibodies to HRP are used to form a soluble semicyclic immune complex consisting of two antibody and three enzyme molecules. The molecular weight of the peroxidase-antiperoxidase or PAP complex is 400-430 kD.

As described above, HRP activity in the presence of an electron donor first results in the formation of an enzyme-substrate complex, and then in the oxidation of the electron donor. The electron donor provides the “driving” force in the continuing catalysis of H₂O₂, while its absence effectively stops the reaction. There are several electron donors that when oxidized, become colored products and are therefore called chromogens. This, and the property of becoming insoluble upon oxidation, make such electron donors useful in immunohistochemistry. The following exemplary substrates/chromogens are contemplated for use with HRP:

3,3′-diaminobenzidine (DAB) produces a brown end product that is highly insoluble in alcohol and other organic solvents. Oxidation of DAB also causes polymerization, resulting in the ability to react with osmium tetroxide, and thus increasing its staining intensity and electron density. Of the several metals and methods used to intensify the optical density of polymerized DAB, gold chloride in combination with silver sulfide appears to be the most successful.

DAB SUBSTRATE SOLUTION

1. Dissolve 6 mg DAB in 10 mL 0.05 M Tris buffer, pH 7.6.

2. Add 0.1 mL 3% hydrogen peroxide. Mix, and filter if precipitate forms.

3. Add solution to tissue and incubate for 3-10 minutes at room temperature.

4. Rinse with distilled water.

5. Counterstain and coverslip with either organicor aqueous-based medium.

3-amino-9-ethylcarbazole (AEC), upon oxidation, forms a rose-red end product that is alcohol soluble. Therefore, specimens processed with AEC must not be immersed in alcohol or alcoholic solutions (e.g., Harris' hematoxylin). Instead, an aqueous counterstain and mounting medium should be used. AEC is unfortunately susceptible to further oxidation and, when exposed to excessive light, will fade in intensity. Storage in the dark is therefore recommended.

AEC SUBSTRATE SOLUTION (recommended for cell smears)

1. Dissolve 4 mg AEC in 1 mL N,N-dimethylformamide.

2. Add 14 mL 0.1 M acetate buffer, pH 5.2 and 0.15 mL 3% hydrogen peroxide.

3. Mix, and filter if precipitate forms.

4. Add solution to tissue and incubate for 5-15 minutes at room temperature.

5. Rinse with distilled water.

6. Counterstain and coverslip with aqueous-based medium.

4-chloro-1-naphthol (CN) precipitates as a blue end product. Because CN is soluble in alcohol and other organic solvents, the specimen must not be dehydrated, exposed to alcoholic counterstains, or coverslipped with mounting media containing organic solvents. Unlike DAB, CN tends to diffuse from the site of precipitation.

p-phenylenediamine dihydrochloride/pyrocatechol (Hanker-Yates reagent) gives a blue-black reaction product that is insoluble in alcohol and other organic solvents. Like polymerized DAB, this reaction product can be osmicated. Varying results have been achieved with Hanker-Yates reagent in immunoperoxidase techniques.

B. Alkaline Phosphatase

Calf intestine alkaline phosphatase (AP) (molecular weight 100 kD) removes (by hydrolysis) and transfers phosphate groups from organic esters by breaking the P-0 bond; an intermediate enzyme-substrate bond is briefly formed. The chief metal activators for AP are Mg++, Mn++ and Ca++.

AP had not been used extensively in immunohistochemistry until publication of the unlabelled alkaline phosphataseantialkaline phosphatase (APAAP) procedure. The soluble immune complexes utilized in this procedure have molecular weights of approximately 560 kD. The major advantage of the APAAP procedure compared to the PAP technique is the lack of interference posed by endogenous peroxidase activity. Because of the potential distraction of endogenous peroxidase activity on PAP staining, the APAAP technique is recommended for use on blood and bone marrow smears. Endogenous alkaline phosphatase activity from bone, kidney, liver and some white cells can be inhibited by the addition of 1 mM levamisole to the substrate solution, although 5 mM has been found to be more effective. Intestinal alkaline phosphatases are not adequately inhibited by levamisole.

In the immunoalkaline phosphatase staining method, the enzyme hydrolyzes naphthol phosphate esters (substrate) to phenolic compounds and phosphates. The phenols couple to colorless diazonium salts (chromogen) to produce insoluble, colored azo dyes. Several different combinations of substrates and chromogens have been used successfully.

Naphthol AS-MX phosphate can be used in its acid form or as the sodium salt. The chromogens Fast Red TR and Fast Blue BB produce a bright red or blue end product, respectively. Both are soluble in alcoholic and other organic solvents, so aqueous mounting media must be used. Fast Red TR is preferred when staining cell smears.

New Fuchsin also gives a red end product. Unlike Fast Red TR and Fast Blue BB, the color produced by New Fuchsin is insoluble in alcohol and other organic solvents, allowing for the specimens to be dehydrated before coverslipping. The staining intensity obtained by use of New Fuchsin is greater than that obtained with Fast Red TR or Fast Blue BB.

FAST RED SUBSTRATE SOLUTION (recommended for cell smears)

1. Dissolve 2 mg naphthol AS-MX phosphate, free acid (Sigma N 4875) in 0.2 mL N,N-dimethylformamide in a glass tube.

2. Add 9.8 mL 0.1 M Tris buffer, pH 8.2.

3. Add 0.01 mL of 1 M levamisole (Sigma L 9756) to block endogenous alkaline phosphatase. (Solution can be stored at 4° C. for several weeks, or longer at −20° C.)

4. Immediately before staining, dissolve 10 mg Fast Red TR salt (Sigma F 1500) in above solution and filter onto slides.

5. Incubate for 10-20 minutes at room temperature.

6. Rinse with distilled water.

7. Counterstain and coverslip with aqueous-based medium.

NEW FUCHSIN SUBSTRATE SOLUTION (recommended for tissue sections)

1. Solution A: Mix 18 mL of 0.2 M 2-amino-2-methyl-1, 3-propanediol (Merck 801464) with 50 mL 0.05 M Tris buffer, pH 9.7 and 600 mg sodium chloride. Add 28 mg levamisole (Sigma L 9756).

2. Solution B: Dissolve 35 mg naphthol AS-BI phosphate (Sigma N 2250) in 0.42 mL N,N-dimethylformamide.

3. Solution C: Under fume hood, mix 0.14 mL 5% New Fuchsin (Sigma N 0638, 5 g in 100 mL 2 N HCl) with 0.35 mL of freshly prepared 4% sodium nitrite (Sigma S 2252, 40 mg in 1 mL distilled water). Stir for 60 sec.

4. Mix Solutions A and B, then add Solution C; adjust to pH 8.7 with HCl. Mix well and filter onto slides.

5. Incubate for 10-20 minutes at room temperature.

6. Rinse with distilled water.

7. Counterstain and coverslip with either organic- or aqueous-based medium.

Additional exemplary substrates include naphthol AS-BI phosphate, naphthol AS-TR phosphate and 5-bromo-4-chloro-3-indoxyl phosphate (BCIP). Other possible chromogens include Fast Red LB, Fast Garnet GBC, Nitro Blue Tetrazolium (NBT) and iodonitrotetrazolium Violet (INT), for example.

VI. Immunodetection Methods

In still further embodiments, the present invention concerns immunodetection methods for binding, purifying, removing, quantifying and/or otherwise generally detecting biological components such as a ligand as contemplated by the present invention. The antibodies prepared in accordance with the present invention may be employed to detect wild-type and/or mutant ligand proteins, polypeptides and/or peptides. As described throughout the present application, the use of wild-type and/or mutant ligand specific antibodies is contemplated. Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. The steps of various useful immunodetection methods have been described in the scientific literature, such as, e.g., Doolittle M H and Ben-Zeev O, 1999; Gulbis B and Galand P, 1993; De Jager R et al., 1993; and Nakamura et al., 1987, each incorporated herein by reference.

In general, the immunobinding methods include obtaining a sample suspected of comprising ligand protein, polypeptide and/or peptide, and contacting the sample with a first anti-ligand antibody in accordance with the present invention, as the case may be, under conditions effective to allow the formation of immunocomplexes.

In terms of antigen detection, the biological sample analyzed may be any sample that is suspected of comprising a wild-type or mutant ligand protein-specific antigen, such as a tissue section or specimen, a homogenized tissue extract, a cell, separated and/or purified forms of any of the above wild-type or mutant ligand protein-containing compositions, or even any biological fluid that comes into contact with the tissue, including blood and/or serum, although tissue samples or extracts are preferred.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any ligand protein antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. U.S. Patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

The anti-ligand antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding agent that has binding affinity for the antibody. In these cases, the second binding agent may be linked to a detectable label. The second binding agent is itself often an antibody, which may thus be termed a “secondary” antibody. The primary immune complexes are contacted with the labeled, secondary binding agent, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding agent, such as an antibody, that has binding affinity for the antibody is used to form secondary immune complexes, as described above. After washing, the secondary immune complexes are contacted with a third binding agent or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

In another embodiment, a biotinylated monoclonal or polyclonal antibody is used to detect the target antigen(s), and a second step antibody is then used to detect the biotin attached to the complexed biotin. In that method the sample to be tested is first incubated in a solution comprising the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution comprising the second step antibody against biotin. This second step antibody is labeled, as for example with an enzyme that can be used to detect the presence of the antibody/antigen complex by histoenzymology using a chromogen substrate. With suitable amplification, a conjugate can be produced that is macroscopically visible.

Another known method of immunodetection takes advantage of the immuno-PCR (Polymerase Chain Reaction) methodology. The PCR method uses a DNA/biotin/streptavidin/antibody complex that is washed out with a low pH or high salt buffer that releases the antibody. The resulting wash solution is then used to carry out a PCR reaction with suitable primers with appropriate controls. In specific embodiments, the enormous amplification capability and specificity of PCR can be utilized to detect a single antigen molecule. Such detection may take place in real-time. For example, the use of quantitative real-time PCR is contemplated.

The immunodetection methods of the present invention have evident utility in the diagnosis and prognosis of conditions, such as various forms of cancer or other conditions that are associated with biomarkers. Here, a biological and/or clinical sample suspected of containing a wild-type or mutant ligand protein, polypeptide, peptide and/or mutant is used. However, these embodiments also have applications to non-clinical samples, such as in the titering of antigen or antibody samples, for example in the selection of hybridomas.

In the clinical diagnosis and/or monitoring of patients with various forms of disease, the detection of ligand mutant, and/or an alteration in the levels of ligand, in comparison to the levels in a corresponding biological sample from a normal subject is indicative of a patient with the disease. However, as is known to those of skill in the art, such a clinical diagnosis would not necessarily be made on the basis of this method in isolation. Those of skill in the art are very familiar with differentiating between significant differences in types and/or amounts of biomarkers, which represent a positive identification, and/or low level and/or background changes of biomarkers. Indeed, background expression levels are often used to form a “cut-off” above which increased detection will be scored as significant and/or positive.

As detailed above, immunoassays, in their most simple and/or direct sense, are binding assays. Certain preferred immunoassays are the various types of enzyme linked immunosorbent assays (ELISAs) and/or radioimmunoassays (RIA) known in the art. Immunohistochemical detection using tissue sections is also particularly useful. However, it will be readily appreciated that detection is not limited to such techniques, and/or western blotting, dot blotting, FACS analyses, and/or the like may also be used.

In one exemplary ELISA, the anti-ligand antibodies of the invention are immobilized onto a selected surface exhibiting protein affinity, such as a well in a polystyrene microtiter plate. Then, a test composition suspected of containing the wild-type and/or mutant ligand protein antigen, such as a clinical sample, is added to the wells. After binding and/or washing to remove non-specifically bound immune complexes, the bound wild-type and/or mutant ligand protein antigen may be detected. Detection is generally achieved by the addition of another anti-ligand antibody that is linked to a detectable label. This type of ELISA is a simple “sandwich ELISA”.

In another exemplary ELISA, the samples suspected of containing the wild-type and/or mutant ligand protein antigen are immobilized onto the well surface and/or then contacted with the anti-ligand antibodies of the invention. After binding and/or washing to remove non-specifically bound immune complexes, the bound anti-ligand antibodies are detected. Where the initial anti-ligand antibodies are linked to a detectable label, the immune complexes may be detected directly. Again, the immune complexes may be detected using a second antibody that has binding affinity for the first anti-ligand antibody, with the second antibody being linked to a detectable label.

Another ELISA in which the wild-type and/or mutant ligand proteins, polypeptides and/or peptides are immobilized, involves the use of antibody competition in the detection. In this ELISA, labeled antibodies against wild-type or mutant ligand protein are added to the wells, allowed to bind, and/or detected by means of their label. The amount of wild-type or mutant ligand protein antigen in an unknown sample is then determined by mixing the sample with the labeled antibodies against wild-type and/or mutant ligand before and/or during incubation with coated wells. The presence of wild-type and/or mutant ligand protein in the sample acts to reduce the amount of antibody against wild-type or mutant ligand protein available for binding to the well and thus reduces the ultimate signal. This is also appropriate for detecting antibodies against wild-type or mutant ligand protein in an unknown sample, where the unlabeled antibodies bind to the antigen-coated wells and also reduces the amount of antigen available to bind the labeled antibodies.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein, or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of about 25° C. to 27° C., or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. Preferably, this will be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

VII. In Situ Hybridization

In situ hybridization is generally carried out on cells or tissue sections fixed to slides. In situ hybridization may be performed by several conventional methodologies (See for e.g. Leitch et al. In situ Hybridization: a practical guide, Oxford BIOS Scientific Publishers, Micropscopy handbooks v. 27 (1994)). In one in situ procedure, fluorescent dyes (such as fluorescein isothiocyanate (FITC) that fluoresces green when excited by an Argon ion laser) are used to label a nucleic acid sequence probe that is complementary to a target nucleotide sequence in the cell. Each cell comprising the target nucleotide sequence will bind the labeled probe, producing a fluorescent signal upon exposure of the cells to a light source of a wavelength appropriate for excitation of the specific fluorochrome used.

Various degrees of hybridization stringency can be employed. As the hybridization conditions become more stringent, a greater degree of complementarity is required between the probe and target to form and maintain a stable duplex. Stringency is increased by raising temperature, lowering salt concentration, or raising formamide concentration. Adding dextran sulfate or raising its concentration may also increase the effective concentration of labeled probe to increase the rate of hybridization and ultimate signal intensity. After hybridization, slides are washed in a solution generally comprising reagents similar to those found in the hybridization solution with washing time varying from minutes to hours depending on required stringency. Longer or more stringent washes typically lower nonspecific background but run the risk of decreasing overall sensitivity.

Probes used in the FISH analysis may be either RNA or DNA oligonucleotides or polynucleotides and may contain not only naturally-occurring nucleotides but their analogs, like digoxygenin dCTP, biotin dcTP 7-azaguanosine, azidothymidine, inosine, or uridine, for example. Other useful probes include peptide probes and analogues thereof, branched gene DNA, peptidometics, peptide nucleic acid (PNA) and/or antibodies, for example.

Probes should have sufficient complementarity to the target nucleic acid sequence of interest so that stable and specific binding occurs between the target nucleic acid sequence and the probe. The degree of homology required for stable hybridization varies with the stringency of the hybridization medium and/or wash medium. Preferably, completely homologous probes are employed in the present invention, but persons of skill in the art will readily appreciate that probes exhibiting lesser but sufficient homology can be used in the present invention (see for e.g. Sambrook, J., Fritsch, E. F., Maniatis, T., Molecular Cloning A Laboratory Manual, Cold Spring Harbor Press, (1989)).

One of skill in the art will appreciate that the choice of probe will depend on the genetic abnormality of interest. Genetic abnormalities that can be detected by this method include, but are not limited to, amplification, translocation, deletion, addition and the like.

Probes may also be generated and chosen by several means including, but not limited to, mapping by in situ hybridization, somatic cell hybrid panels, or spot blots of sorted chromosomes; chromosomal linkage analysis; or cloned and isolated from sorted chromosome libraries from human cell lines or somatic cell hybrids with human chromosomes, radiation somatic cell hybrids, microdissection of a chromosome region, or from yeast artificial chromosomes (YACs) identified by PCR primers specific for a unique chromosome locus or other suitable means like an adjacent YAC clone. Probes may be genomic DNA, cDNA, or RNA cloned in a plasmid, phage, cosmid, YAC, Bacterial Artificial Chromosomes (BACs), viral vector, or any other suitable vector. Probes may be cloned or synthesized chemically by conventional methods. When cloned, the isolated probe nucleic acid fragments are typically inserted into a vector, such as lambda phage, pBR322, M13, or vectors containing the SP6 or T7 promoter and cloned as a library in a bacterial host. [See for e.g. Sambrook, J., Fritsch, E. F., Maniatis, T., Molecular Cloning A Laboratory Manual, Cold Spring Harbor Press, (1989)].

Probes are preferably labeled, such as with a fluorophor, for example. Examples of fluorophores include, but are not limited to, rare earth chelates (europium chelates), Texas Red, rhodamine, fluorescein, dansyl, Lissamine, umbelliferone, phycocrytherin, phycocyanin, or commercially available fluorophors such SPECTRUM ORANGE™ and SPECTRUM GREEN™ and/or derivatives of any one or more of the above. Multiple probes used in the assay may be labeled with more than one distinguishable fluorescent or pigment color. These color differences provide a means to identify the hybridization positions of specific probes. Moreover, probes that are not separated spatially can be identified by a different color light or pigment resulting from mixing two other colors (e.g., light red+green=yellow) pigment (e.g., blue+yellow=green) or by using a filter set that passes only one color at a time.

Probes can be labeled directly or indirectly with the fluorophor, utilizing conventional methodology known to one with skill in the art.

VIII. Real-Time Detection

In specific embodiments of the invention, one or more methods employ real-time detection. Any suitable real-time detection method may be used, but in specific embodiments the real-time detection includes polymerase chain reaction. In specific embodiments of real-time polymerase chain reaction, the real-time detection collects data in the exponential growth phase. An advantage to using real-time PCR is that an increase in the signal is directly proportional to the number of amplified molecules that are produced. Detection capacity is highly sensitive, including down to a 2-fold change in certain embodiments. Commercial kits may be employed, such as from Applied Biosystems (Foster City, Calif.) or Cepheid (Sunnyvale, Calif.), for example.

Other potential embodiments include the real-time measurement of any detection system which generates a changing signal over time. For example, in the QUELI procedure where and insoluble colored signal is generated, the ELISA plate reader could be programmed to measure changes in the signal over time by repeatedly taking measurement in the same well, etc.

IX. Kits and Compositions

Also provided by the invention are kits for use in the practice of the present invention as disclosed herein. Such kits may comprise containers, each with one or more of the various reagents (typically in concentrated form) utilized in the methods, including, for example, one or more binding agents, already attached to a marker or optionally with reagents for coupling a binding agent to a marker or nucleic acid molecule (as well as the marker itself); buffers, the appropriate nucleotide triphosphates (e.g. dATP, dCTP, dGTP, dTTP, dUTP, ATP, CTP, GTP and UTP), reverse transcriptase, DNA polymerase, RNA polymerase, and one or more sequence-specific or degenerate primers for use in detection of nucleic acid molecules by amplification; and/or reagents and instrumentation for the isolation (optionally by microdissection) to support the practice of the invention. A label or indicator describing, or a set of instructions for use of, kit components in a ligand detection method of the present invention, will also be typically included, where the instructions may be associated with a package insert and/or the packaging of the kit or the components thereof.

In still further embodiments, the present invention concerns immunodetection kits for use with the immunodetection methods described above. As the antibodies are generally used to detect wild-type and/or mutant proteins, polypeptides and/or peptides, the antibodies will preferably be included in the kit. The immunodetection kits will thus comprise, in suitable container means, a first antibody that binds to a wild-type and/or mutant protein, polypeptide and/or peptide, and/or optionally, an immunodetection reagent and/or further optionally, a wild-type and/or mutant protein, polypeptide and/or peptide.

The immunodetection reagents of the kit may take any one of a variety of forms, including those detectable labels that are associated with and/or linked to the given antibody. Detectable labels that are associated with and/or attached to a secondary binding ligand are also contemplated. Exemplary secondary ligands are those secondary antibodies that have binding affinity for the first antibody.

Further suitable immunodetection reagents for use in the present kits include the two-component reagent that comprises a secondary antibody that has binding affinity for the first antibody, along with a third antibody that has binding affinity for the second antibody, the third antibody being linked to a detectable label. As noted above, a number of exemplary labels are known in the art and/or all such labels may be suitably employed in connection with the present invention.

The kits may further comprise a suitably aliquoted composition of the wild-type and/or mutant protein, polypeptide and/or polypeptide, whether labeled and/or unlabeled, as may be used to prepare a standard curve for a detection assay. The kits may contain antibody-label conjugates either in fully conjugated form, in the form of intermediates, and/or as separate moieties to be conjugated by the user of the kit. The components of the kits may be packaged either in aqueous media and/or in lyophilized form.

The container means of the kits will generally include at least one vial, test tube, flask, bottle, syringe and/or other container means, into which the antibody may be placed, and/or preferably, suitably aliquoted. The kits of the present invention will also typically include a means for containing the antibody, antigen, and/or any other reagent containers in close confinement for commercial sale. Such containers may include injection and/or blow-molded plastic containers into which the desired vials are retained.

For example, in one embodiment of the invention, a kit will assess comprehensive panels of molecules (e.g. clinically relevant prognostic and predictive factors in cancer) in broad clinical and research settings.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Experimental Design

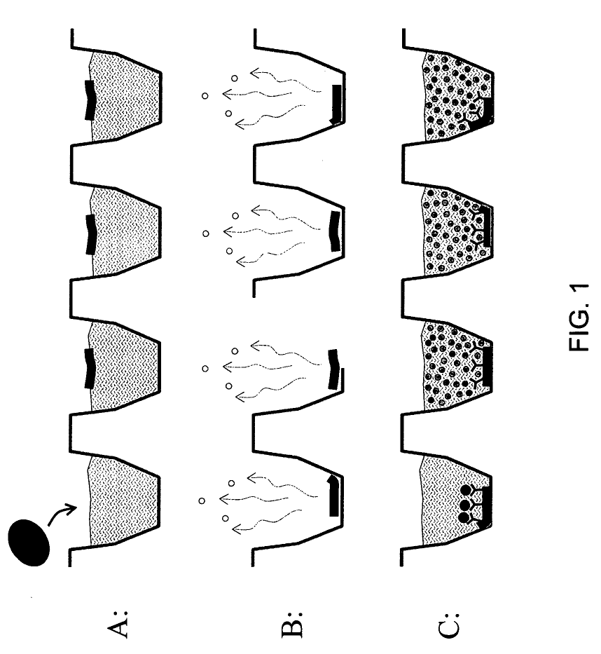

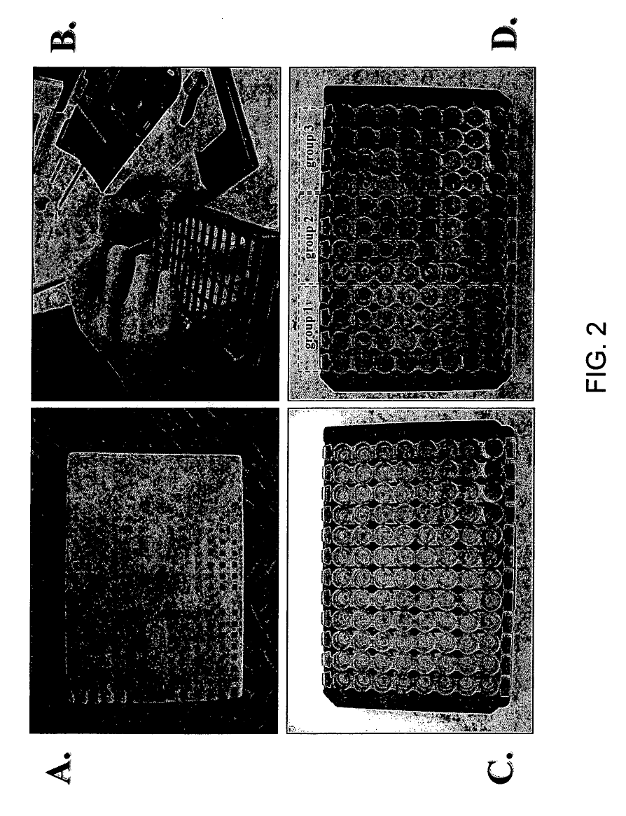

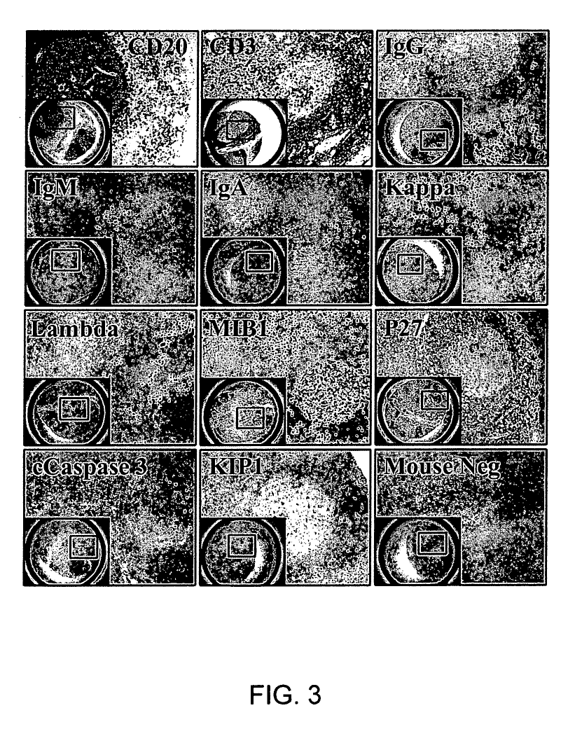

Utilizing routine microtomy, serial sections (usually 3-4 um thick) of a core (usually 2-3 mm diameter) of FFPET tissue were placed individually into water-filled wells of a microtiter plate. The water was evaporated, allowing the sections to descend and adhere to the bottom of the wells. The sections were then deparaffinized, followed by heat-induced epitope unmasking (e.g. in a thermocycler at 110° C. for 30 minutes). Thereafter, the plate was subjected to a series of reagents and washes following a standard ELISA-like procedure. At the primary antibody step of the procedure, a primary antibody was plated into 4 consecutive wells. The number, specificity, and type (species) of antibodies can be mixed and varied depending on the application, but in a typical 96-well plate, up to different 24 antibodies were quantitatively evaluated in triplicate. At the chromogen step of the procedure, the first well for each antibody received an insoluble chromogen (H₂O₂/DAB) and the remaining three received a liquid-phase soluble chromogen (H₂O₂/TMB) that was allowed to react continually until a prominent colored signal was generated (usually 30 minutes). The continuous generation of colored product greatly amplified the signal until the reaction was stopped. The wells with DAB received a hematoxylin counterstain and a liquid cover-slip, enabling direct microscopic visualization. The wells with TMB were quantified by measuring optical density (OD) at 450 nm wavelength with a standard microplate reader. At least three types of information were produced as follows: (1) the type of tissue/cell expressing the protein; (2) the proportion of tissue/cells expressing the protein; and (3) the relative linear quantity of the protein being expressed (range ˜3 logs/base 10). FIGS. 1-3 and Table 1 illustrate and describe these procedures in more detail.

### Example 2

### Screen of Human Tonsil FFPET

An assay was performed on intact human tonsil (FFPET core) to assess the capacity of the assay for detecting multiple targets on a single 96-well plate. The estimated percentage of positive cells was determined by direct microscopic visualization of tissue in wells receiving insoluble chromogen. The optical density of the cells was measured in triplicate using a standard microplate reader. The signal from the soluble chromogen is highly amplified (range ˜3 logs/base 10) in a linear manner, and is much more quantitative than the direct microscopic visualization. The statistical significance was determined using a two-tailed unpaired test of comparisons between the level of protein and background with species-matched non-immune antibodies as a negative control. The assay can detect significant levels of protein expression from proteins expressed from very rare cells, such as p53 and cleaved-caspase 3, which are both expressed at very low levels in <1% of the cells sampled.

TABLE 1
Summary of results from an assay assessing 24 exemplary
antibodies on intact human tonsil (FFPET core) measured
simultaneously in one 96-well microtiter plate

| Antigen/Antibody | | Avg OD | P-value |
|---|---|---|---|
| (Species) | Est % Pos Cells | (triplicate) | (vs. Neg) |
| Neg control/non-immune | 0% | 0.069 | NA |
| IgGs (M) | | | |
| Neg control/non-immune | 0% | 0.022 | NA |
| IgGs (R) | | | |
| P53 (M) | <1% | 0.196 | <0.0001 |
| IgA heavy chain (R) | 1% | 0.200 | <0.0001 |
| Leu-M1 (M) | 5% | 0.251 | <0.0001 |
| Mac387 (M) | 5% | 0.323 | <0.0001 |
| IgM heavy chain (M) | 5% | 0.379 | 0.0001 |
| Cleaved-caspase 3 (R) | <1% | 0.383 | 0.0006 |
| Mitosin (M) | 5% | 0.416 | <0.0001 |
| KP1 (M) | 5% | 0.505 | <0.0001 |
| Lambda light chain (R) | 10% | 0.630 | <0.0001 |
| Topoisomerase II (M) | 5% | 0.694 | <0.0001 |
| IgG heavy chain (R) | 10% | 0.935 | 0.0004 |
| P27 (M) | 75% | 1.510 | <0.0001 |
| CD31 (M) | 10% | 1.596 | <0.0001 |
| Kappa light chain (R) | 15% | 1.672 | 0.0002 |
| Cytokeratin (M) | 5% | 1.760 | <0.0001 |
| MIBI (M) | 20% | 1.932 | 0.0003 |
| Bcl2 (M) | 80% | 1.993 | 0.0001 |
| Tubulin (M) | 25% | 2.219 | <0.0001 |
| CD3 (R) | 40% | 2.743 | <0.0001 |
| CD45 (M) | 95% | 3.019 | <0.0001 |
| CD20 (M) | 60% | 3.457 | <0.0001 |

### REFERENCES

All patents and publications mentioned in the specifications are indicative of the levels of those skilled in the art to which the invention pertains. All patents and publications are herein incorporated by reference to the same extent as if each individual publication was specifically and individually indicated to be incorporated by reference.

Bondi A et al. Histochemistry 1982; 76:153-158.

Clark C A et al. J. J Histochem Cytochem 1982; 30:27-34.

Cordell J L et al. J Histochem Cytochem 1984;32:219-229.

DakoCytomation Specifications No. K0597, K0598, K0599, K0624, K0698.

DakoCytomation Specifications No. K3461, K3465, K3467.

Gay et al. J Histochem Cytochem 1984; 32:447-451.

Gown A M In DeLellis R A (ed) Advances in Immunohistochemistry, New York: Raven Press, 1988, pp 31-45.

Mason D Y et al. J Cancer Res Clin Oncol 1981; 101:13-22.

Newman G R et al. J Microscopy 1983; 132; RP1-2.

Newman G R et al. J Microscopy 1983;132:RP1-2.

Ponder B A and Wilkinson M M J. Histochem Cytochem 1981;29:981-984.

Sternberger L A. Immunocytochemistry (2nd ed). New York: Wiley, 1979.

Although the present invention and its advantages have been described in detail, it should be understood that various changes, substitutions and alterations can be made herein without departing from the invention as defined by the appended claims. Moreover, the scope of the present application is not intended to be limited to the particular embodiments of the process, machine, manufacture, composition of matter, means, methods and steps described in the specification. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

We claim:

1. A method for quantitatively detecting a ligand in a sample comprising:
contacting the sample with a binding agent capable of binding the ligand;
contacting a section of the sample with an insoluble indicator, wherein the insoluble indicator allows for microscopic visualization of the binding agent;
contacting another section of the sample with a soluble indicator, wherein the soluble indicator allows for quantitative measurement of the binding agent; and
correlating the microscopic visualization of the binding agent with the quantitative measurement of the binding agent in order to quantitatively detect the ligand in the sample.

2. The method of claim 1 wherein said sample is a tissue section, a cytospin, a cell smear, or a mixture thereof.

3. The method of claim 2, wherein the tissue section is embedded in a solid medium.

4. The method of claim 3, wherein the solid medium is paraffin or plastic.

5. The method of claim 1 wherein the binding agent comprises at least one antibody.

6. The method of claim 5, wherein the binding agent comprises a primary antibody and a secondary antibody.

7. The method of claim 6, wherein the secondary antibody is enzyme-linked.

8. The method of claim 7, wherein the enzyme is horseradish peroxidase or alkaline phosphatase.

9. The method of claim 6, wherein the primary antibody is biotinylated.

10. The method of claim 1 wherein the quantitative measurement of the soluble indicator comprises detection of optical density, fluorescence, luminescence, or a combination thereof.

11. The method of claim 1 wherein the quantitative measurement of the soluble indicator comprises real-time detection.

12. The method of claim 1 wherein the soluble indicator is a calorimetric, chemifluorescent, or chemiluminescent enzyme substrate.

13. The method of claim 1 wherein the soluble indicator is a chromogen.

14. The method of claim 1 wherein the soluble indicator is selected from the group consisting of 3-3′,5,5′-tetramethylbenzidine, O-dianisidine, and p-nitrophenyl phosphate.

15. The method of claim 1 wherein the insoluble indicator is a chromogen.

16. The method of claim 1 wherein the insoluble indicator is selected from the group consisting of 3,3′-diaminobenzidine, 3-amino-9-ethylcarbazole, 4-chloro-1-naphthol, p-phenylenediamine, dihydrochloride/pyrocatechol, naphthol AS-MX phosphate, new fuchsin, AS-BI phosphate, naphthol AS-TR phosphate and 5-bromo-4-chloro-3-indoxyl phosphate (BCIP) Fast Red LB, Fast Garnet GBC, Nitro Blue Tetrazolium (NBT) and iodonitrotetrazolium Violet (INT).

17. The method of claim 1, wherein the binding agent comprises a nucleic acid probe.

18. The method of claim 1 wherein the ligand is a polypeptide, a nucleic acid, a lipid, a carbohydrate, or a portion or domain or epitope thereof.

19. The method of claim 1 wherein the ligand is selected from the group consisting of p53, Leu-M1, Mac387, cleaved-caspase 3, mitosin, KP1, topoisomerase II, p27, CD31, cytokeratin, MIBI, Bcl2, tubulin, CD3, CD45, and CD20.

20. The method of claim 1, wherein the sample comprises a patient sample having a tumor-associated marker.

21. The method of claim 20, wherein said sample provides information for a diagnosis, treatment, or both of a subject.

22. The method of claim 21, wherein the subject has cancer or is suspected of having cancer.

23. A method of screening a sample for the presence of a tumor-associated marker comprising:
contacting the sample with a binding agent capable of binding the tumor-associated marker;
contacting a section of the sample with an insoluble indicator, wherein the insoluble indicator allows for microscopic visualization of the binding agent;
contacting another section of the sample with a soluble indicator, wherein the soluble indicator allows for quantitative measurement of the binding agent;
and correlating the microscopic visualization of the binding agent with the quantitative measurement of the binding agent in order to quantitatively detect the tumor-associated marker in the sample.

24. A method for quantitative detection of a ligand in a formalin-fixed paraffin-embedded biological tissue sample comprising:
deparaffinizing the sample;
heating the sample;
detecting the ligand by an enzyme-linked immunoassay in a section of the sample;
detecting the ligand by immunohistochemical staining in another section of the sample; and
quantitatively detecting the ligand by correlating the results of the immunohistochemical staining with the results of the enzyme-linked immunoassay.

25. A kit housed in a suitable container, said kit suitable for performing the method of claim 1, comprising a soluble indicator, an insoluble indicator, and a binding agent.
